(19) United States
(12) Patent Application Publication
(10) Pub. No.: US 2002/0155503 A1
(43) Pub. Date: Oct. 24, 2002

(54) Method for mapping the active sites bound by enzymes that covalently modify substrate molecules

(75) Inventors: Jonathan Clark, Cambridge (GB); Alan Lamont, Herts (GB); David Williams, Herts (GB)

Correspondence Address: NIXON & VANDERHYE P.C., 8th Floor, 1100 North Glebe Road, Arlington, VA, 22201-4714, US

(73) Assignee: Mimetrix Limited

(21) Appl. No.: 10/020,436

(22) Filed: Dec. 18, 2001

Related U.S. Application Data
(63) Continuation of application No. 09/530,431, filed on Jul. 26, 2000, now abandoned.

(30) Foreign Application Priority Data
Oct. 30, 1997 (GB) 9722818.3
Oct. 30, 1998 (GB) PCT/GB98/03259

(51) Int. Cl. G01N 33/53; C12P 21/04; G01N 33/543

(52) U.S. Cl. 435/7.1; 436/518

(57) ABSTRACT

This invention provides for the active site mapping of enzymes which catalyse covalent modification including, but not limited to phosphorylation, acylation, dephosphorylation in which a fixed residue (known as the catalytic residue) such as a tyrosine, serine, threonine, histidine, aspartic acid residue or any other residue containing an appropriate side chain is modified. Mapping of protein kinases is exemplified. The method of the invention has an additional level of complexity over and above that of the self-deconvoluting libraries described in W097/42216. This involves making a library of smaller libraries (referred to as subsets) where a fixed residue is moved stepwise through the sequence of amino acids or other groups (such as peptidomimetics). Using 5 subsets of libraries of peptides of 5 amino acids allows the mapping of a sequence of 9 amino acids. In general one could carry out the invention using n subsets of n-mer peptides so as to provide mapping data for the residues from −(n−1) to +(n−1) either side of the active site. Thus in general the length of the mapped sequence would be (2n)−1. In this invention there is no need to separate modified from unmodified sequences because of the self deconvoluting nature of the library. The assay screen produces a series of hits, the patterns of which reveal the unique sequences in each well. This enables a pattern of substrate preferences to be determined for any enzyme. The unique sequences obtained using this invention can be used to provide substrates for high throughput assays and provide detailed information about the active site to aid rational drug design. This invention can also be used as an inhibitor library to screen against known modifying enzymes where a known substrate exists and can be set up in an assay format.

years as more information from gene sequenceing databases becomes available. Functionally, these molecules are intracellular enzymes that play key roles in cell growth, differentiation and inter-cell communication. Aberrant protein kinase activity has been implicated in many disease states including, several forms of cancer and severe-combined immunodeficiency disease (Barker et al, 1997; Lehtola et al; Arpaia et al 1994; Elder et al, 1995; Roifman, 1995). Similarly, activation of protein kinase activity within nononuclear cells is required to drive the cytokine production which underlies many autoimune diseases (Lee et al, 1994). Thus, inhibitor compounds capable of specifically inactivating certain critical kinases may have considerable therapeutic benefit in a number of clinical diseases.

All tyrosine and serine/threonine protein kinases have a region of approximately 300 amino acids known as the catalytic subunit which has evolved from a common ancestor kinase (Hanks and Quinn, 1991). Crystal structure determination of several kinases has shown that they all have a common bi-lobal structure (Wilson et al, 1996; Zhang et al, 1994; Xu et al, 1997). The amino-terminal part of the subunit encodes a small lobe responsible for the binding of ATP, whereas the carboxy-terminal residues encode a larger lobe important for protein substrate binding. In the tertiary structure of the active kinase, both the ATP and the protein substrate binding sites are brought together allowing transfer of the ATP γ-phosphate to the amino acid acceptor on the protein substrate. The protein/peptide binding groove stretches across the face of the large lobe between two α-helices and under the small lobe. This groove therefore contains the residues important for defining the substrate specificity of the kinase.

Many protein kinases are arranged in kinase cascades within the cell, providing the ability for signal amplification in post-transduction pathways. This amplification relies on the upstream kinase specifically activating its downstream parmeter. For this reason, protein kinases have developed remarkable substrate specificities which prevent unwanted crosstalk between different kinase cascades. This substrate specificity can be exploited in the design of selective protein kinase inhibitors.

A technique has recently been developed by L. Cantley's laboratory to provide, consensus peptide protein kinase substrate information (WO 95/18823. Songyang et al, 1994). First, a degenerate library of peptides with a phospho-acceptor such as tyrosine or serine/threonine flanked by amino acids on each side is synthesised. A preferred number of degenerate residues on each side of the phosphorylation site is four (corresponding to positions −1, −2, −3, −4, +1, +2, +3, +4) relative to the phosphorylated residue. Thus the library consists of peptides having a length of nine amino acids. The library is then phosphorylated by the protein kinase of interest and phosphorylated peptides isolated from the non-phosphorylated peptides by DEAE-sephacel and ferric chelation chromatography. The phosphopeptide mixture is then sequenced and the frequency of each amino acid at every position assessed to give a preferred substrate sequence. These studies have yielded consensus substrate information, but do not allow a detailed analysis of particular preferences for neighbouring residue interactions as pools of peptides are examined. Furthermore, this type of analysis may not show up rare good substrates which could be hidden by the presence of numerous poor substrates in the peptide pool. By this method individual peptides can never be identified as individual sequences, the result is that an average picture of substrate specificity is reached. Part of the problem is that each individual peptide is represented at such a low level, and many inevitably will not even be present. The results from Cantley's method do not represent individual peptides but a consensus picture of protein kinase substrate specificity.

Filamentous phage expressing gene III-linked degenerate peptide sequences have also been used to generate substrate information (Schmitz et al, 1996; Dente et al, 1997), however this method is labour intensive and does not allow the use of unnatural amino acids or peptidomimetics. Substrate information can also be obtained from knowledge of the physiological kinase substrates. This approach is limited and previous attempts to utilise this information for the design of successful therapeutic cell permeable protein kinase inhibitors has failed (Kemp et al, 1991).

We believe that identification of detailed substrate characteristics can intimately map the substrate binding groove and provide information that can lead to the design of enzyme inhibitor molecules. For the reasons described above, there are no current methods for obtaining this information. Therefore, we have invented a method of using small molecules, in a self deconvoluting library format, to probe a larger active site by positional scanning of a target group. This method is rapid, not labour intensive and results in the identification of discrete sequences.

### SUMMARY OF INVENTION

This invention provides for the active site mapping of enzymes which catalyse-covalent modification including, but not limited to phosphorylation, acylation, dephosphorylation in which a fixed residue (hereafter known as the catalytic residue) such as a tyrosine, serine, threonine, histidine, aspartic acid residue or any other residue containing an appropriate side chain is modified. The method of the invention has an additional level of complexity over and above that of the self-deconvoluting libraries described in WO97/42216 (the content of which is incorporated herein by reference, where legally permissible).

This involves making a library of smaller libraries (referred to as sub-sets) where a fixed residue is moved stepwise through the sequence of amino acids or other groups (such as peptidomimetics [any compound that can be added to the substrate or inhibitor chain]). The result is that in each sub-set of the library the fixed residue is found in a different position. For example, in a library using four variable positions, five sub-sets in each library have to be made, ZXXXX, XZXXX, XXZXX, XXXZX, and XXXXZ where Z is the fixed residue and X are the four variable residues. We recognise that there may be a need in certain circumstances for further invariant residues, however these would occupy fixed positions and would not affect either the scanning or the self-deconvoluting of the libraries. The invariant residue(s) might be fixed in position relative to the modifiable residue Z or may be fixed in position relative to the overall motif sequence. Additional fixed residues can be added if desired, or one of the variable residues can be made invariant. In the later case the library would be a small part of the libraries described here. Cases where it is desirable to include one or more fixed residues include libraries required to look at enzymes which always require another invariant residue in another position. However, in cases where two faced residues are required, and they are both modified, it can be desirable to include this residue in one of the variable positions (i.e. make it one of the residues chosen in a variable position). The reason for this is that the sequence of events (the order in which the two residues become modified) can then also be probed by this scanning library technique. In this case it may also be beneficial to make an additional library in which the fixed residue is not present at all, corresponding to the library XXXX. We would therefore have a library of six sub-sets. These modifications are within the scope of the invention and would be recognised by someone skilled in the art.

It can be readily seen that by combining the data from each library sub-set, the residues from −4 to +4 either side of the catalytic residue can be mapped:

A-B-C-D-Z

B-C-D-Z-E

C-D-Z-B-F

D-Z-E-F-G

Z-E-F-G-H

The mapped sequence would therefore be A-B-C-D-Z-E-F-G-H.

The above example using 5 subsets of libraries of peptides of 5 amino acids allows the mapping of a sequence of 9 amino acids. In general one could carry out the invention using n subsets of n-mer peptides so as to provide mapping data for the residues from −(n−1) to +(n−1) either side of the active site. Thus in general the length of the mapped sequence would be (2n)−1.

Where the residue type at any given, position relative to the fixed residue is similar in different subsets, the data can be used in an additive manner. For example, if an aromatic residue is required adjacent to the fixed residue, then any sequences which contain this feature in any of the library subsets can be considered in an additive way.

In this invention there is no need to separate modified from unmodified sequences because of the self deconvoluting nature of the library. The assay screen produces a series of hits, the patterns of which reveal the unique sequences in each well. This enables a pattern of substrate preferences to be determined for any enzyme.

The unique sequences obtained using this invention can be used to provide substrates for high throughput assays and provide detailed information about the active site to aid rational drug design.

This invention can also be used as an inhibitor library to screen against known modifying enzymes where a known substrate exists an a can be set up in an assay format. Those skilled in the art would realise that by replacing the fixed residue with a suitable compound that is not modified an inhibitor library can be constructed. For example if a modifiable fixed tyrosine were to be changed to a tyrosine derivative residue that cannot be phosphorylated, such as halogenated tyrosine, dopamine, or tyrosine substituted by aromatic compounds, then an inhibitor library will be formed. This could allow the more direct identification of prototype inhibitors of enzymes for rational drug design.

Use of these libraries could be extended to other systems where a defined endpoint is desired, but the target enzyme is unknown. Such examples could include, but are not limited to, bacterial lysis in growing cultures or inhibiting phosphorylation of transcription factors in cell lysates.

In one embodiment of this invention the sequences identified by this method are considerably smaller than have previously been reported for library screens on protein kinase substrates, which makes them more amenable to computer modelling and drug design. Furthermore, this methodology provides information about the relative relationships between neighbouring residues of active substrates; information which is not available from a straightforward oriented degenerate peptide library approach used by Cantley (Songyang et al, 1994). Thus, this novel methodology provides a significant improvement in the quality of substrate based information that is achievable, in comparison to that produced from previously described methods.

This invention allows data to be obtained from single peptide rankings which could be used to rationally design sets of enzyme inhibitor molecules which compete with the physiological substrate for binding to the active site of the enzyme.

### DESCRIPTION OF THE DRAWINGS

FIG. 1. Design of protein tyrosine kinase library. Each peptide consists of a biotin tag, an epsilon amino hexanoic acid spacer and 5 amino acids, including a phosphorylatable tyrosine residue. Each of the amino acid positions A-H is varied as described. For example, A1-10 means that position A is varied using 10 defined amino acids.

FIG. 2. Best substrates identified by screening tyrosine library sub-sets 1 to 5 against ZAP-70 protein tyrosine kinase. The protein tyrosine kinase library described in FIG. 1 was phosphorylated for 30 minutes at 30° C. using the catalytic domain of human ZAP-70. Peptides were captured using strepavidin-coated 96 well plates and phosphotyrosine detected using anti-phosphotyrosine antibody, anti mouse IgG-HRP and tetramethylbenzidine (see experimental methods). Best substrates were identified as those which gave the highest amount of phosphate incorporation.

FIG. 3. Km Determination of Biotin-εAHA-DEEDYFE(Nle) [SEQ ID NO. 3]. The catalytic domain of human ZAP-70 was used to phosphorylate varying concentrations of peptide for 10 minutes at 30° C. in the presence of ³³P-γ-ATP. Peptide capture was performed using strepavidin filter plates, scintillation fluid added, and counting performed using a beta-counter (see experimental methods). Samples were assayed in triplicate.

### DESCRIPTION OF THE INVENTION

This invention provides for the active site mapping of enzymes which catalyse covalent modification including, but not limited to, phosphorylation, acylation, dephosphorylation in which a residue such as a tyrosine, serine, threonine, histidine, aspartic acid or any other residue containing an appropriate side chain is modified.

Thus, the invention provides a method for determining an amino acid sequence motif or a peptidomimetic sequence motif containing an active site capable of being bound by an enzyme which catalyses covalent modification of a substrate molecule, comprising;

a) contacting the enzyme with a library consisting of a number of oriented degenerate library subsets of molecules, each subset comprising unmodified degenerate motif sequences each having n residues and each having a modifiable residue at a different fixed non-degenerate position, under conditions which allow for modification of molecules which are a substrate for the enzyme;

b) allowing the enzyme to modify modifiable residues in library subsets containing molecules having an active substrate site for the enzyme;

c) deconvoluting the oriented degenerate library subsets of the library, in situ without separating modified from unmodified molecules, so as to reveal the sequence of any motif which has been modified by covalent binding of the enzyme;

wherein each library subset is of formula (I)

(Xaa)ₓZaa(Xaa)_{y} (I)[SEQ ID No. 1]

wherein

Zaa is a non-degenerate modifiable natural or unnatural amino acid residue or peptidomimetic;

Xaa is any natural or unnatural amino acid residue or peptidomimetic;

x and y are each independently 0 or an integer;

(x+y)=(n−1); and

n=an integer from 3 to 8, preferably 5.

This invention can be applied for instance to a protein tyrosine kinase in order to exemplify the technology. It provides a rapid method of identifying discrete protein kinase substrate sequences which allows pharmacophore generation and design of active site inhibitors. This invention can also be used to directly identify protein kinase inhibitor molecules. General formula: (Xaa)ₓ Tyr (Xaa)_{y} [SEQ.ID No. 2].

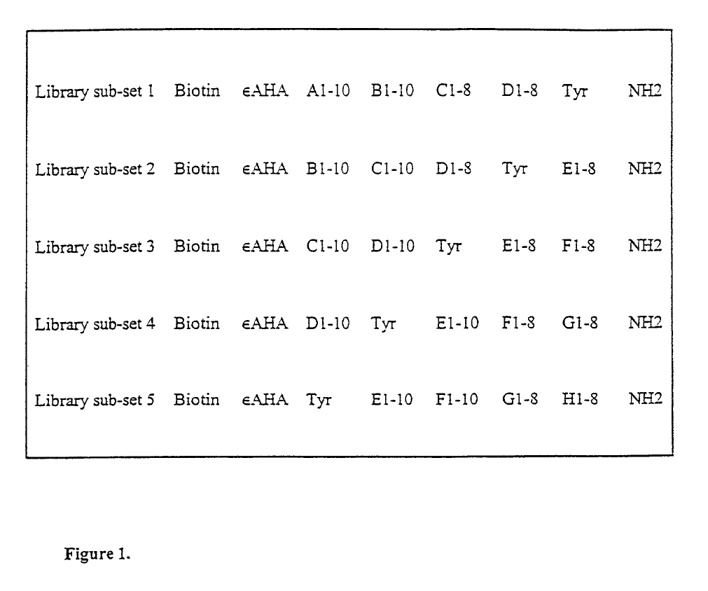

In the first exemplification of this invention, a recombinant form of the human ZAP-70 enzyme was used in an in vitro phosphorylation reaction to phosphorylate the five substrate sub-libraries which scan the sequence −4 to +4 around a central tyrosine residue (FIG. 1). The libraries were arranged in 96 well microtiter plate format with pools of 20 peptides in each microtiter well. However, those skilled in the art will realise that the library can be constructed on any scale. For example the library Sub-Set can be miniaturised on a “chip” scale or constructed on a large bulk scale depending on the requirements of the library.

Library peptides were made with biotin tags, which allowed peptide capture on strepavidin-coated microtiter plates. Detection of phosphotyrosine was achieved using anti-phosphotyrosine antibody detection in an ELISA assay using tetramethylbenzidine substrate and recording absorbance at 450 nm. Background absorbance readings of 0.1 to 0.2 were recorded while the highest substrate peptide value was 1.5. Deconvolution of the hit peptides was performed as described in WO 97/42216. Clear defined substrates were deconvoluted in library sub-sets 1 to 4, but not in 5. This probably reflects the absolute requirement of ZAP-70 for an amino acid residue in the −1 position.

For the purpose of this exemplification, the peptides used were tagged with d-Biotin and a linker (epsilon amino hexanoic acid or some other spacing group). In principle any tag and linker can be used, although this invention also provides that a tag and linker does not have be present if mass spectroscopy, for example, is used to identify the peptide hits. The purpose of the tag is solely to enable capture of all of the peptides (whether modified or not) so that excess reagents can be washed away. The reporting systems to detect peptide modification can include, but are not limited to, antibody recognition, radioactive assay or mass spectroscopy.

In the library used to exemplify the invention, a Biotin tag was chosen because we believed that this would give improved results. The reasoning for this choice of tag was because of the high level of positively charged groups on the enzyme in the area in which the tag sits. This charged area would cause unfavourable interactions with tags more commonly use by others in the field, such a poly-lysine or poly-arginine. We would expect this reasoning to be applicable to any enzyme which binds a highly negatively charged molecule such as but not limited to ATP, close to the peptide binding site.

Tags are preferably non peptide, with as little charge, either positive or negative, as possible. Biotin is a good example of this. The aim is to minimise the interactions of the tag with the protein so the resultant hits are largely due to the binding of the peptides rather than reflecting the binding of the tag. The best method of all if this argument is applied to its logical conclusion would be to not use a tag at all and use mass spectroscopy to identity the peptides. However, currently this approach is of limited value due to the time taken to run and analyse a library of the size used here to exemplify the invention.

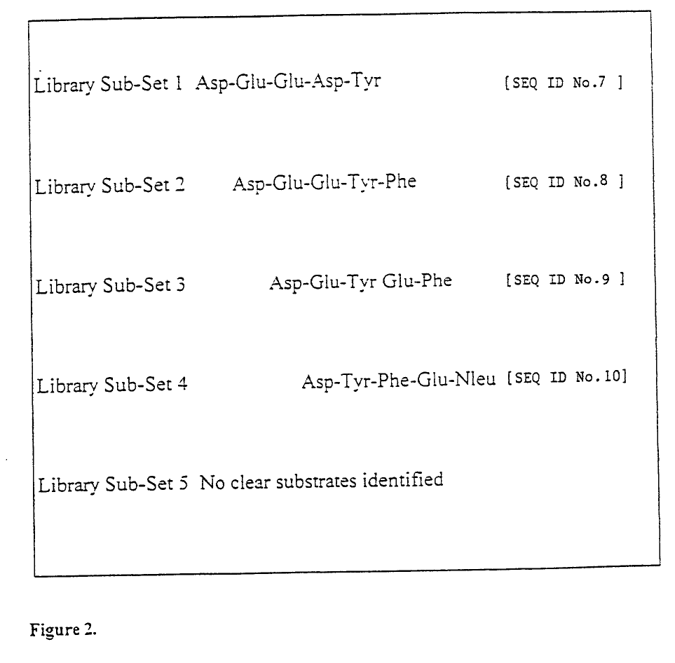

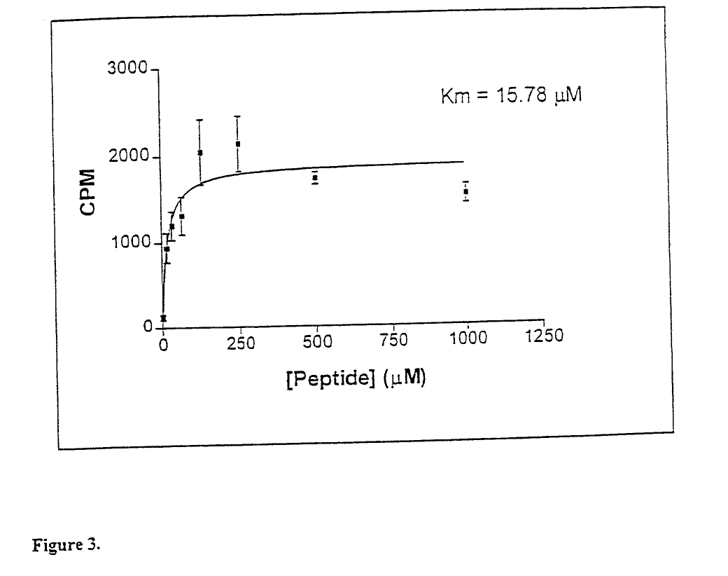

The results obtained from the library screen clearly demonstrated amino acid residues preferred by the protein kinase at each of the −4 to +4 sites (FIG. 2). The 5 mer peptides overlapped to give information on amino acid preference at each of the binding positions −4 to +4. To confirm this a consensus peptide, Biotin-εAHA-DEEDYFE(Nle) [SEQ ID No. 3], representing the best −4 to +4 amino acids was made and tested as a substrate (FIG. 3). This substrate gave a Km against ZAP-70 of 15.79 μM, which is better than the best ZAP-70 substrate described in the literature, a longer peptide of 14 amino acids with a tag of 3 arginines and a Kₘ of 29 μM (Wandenburg et al, 1996).

In the second application of this invention, a recombinant form of the human Syk enzyme was used in an in vitro phosphorylation reaction to phosphorylate the five substrate sub-libraries which scan the sequence to −4 to +4 around a central tyrosine residue, as previously performed for the ZAP-70 library. Detection of phosphotyrosine was achieved using anti-phosphotyrosine antibody detection in an ELISA assay using tetramethylbenzidine substrate and recording absorbance at 450 nm. Background absorbance readings of 0.10 were recorded while the highest substrate peptide value was 1.46. Deconvolution of the hit peptides was performed as described in WO 97/42216. Clear defined substrates were deconvoluted in all library sub-sets.

In the third application of this invention, a recombinant form of the human CSK enzyme was used in an in vitro phosphorylation reaction to phosphorylate the five substrate sub-libraries which scan the sequence 4 to +4 around a central tyrosine residue, as previously performed for the ZAP-70 library. Detection of phosphotyrosine was achieved using anti-phosphotyrosine antibody detection in an ELISA assay using tetramethylbenzidine substrate and recording absorbance at 450 nm. Background absorbance readings of 0.04 were recorded while the highest substrate peptide value was 0.22. Deconvolution of the hit peptides was performed as described in WO 97/42216. Clear defined substrates were deconvoluted in all library sub-sets.

In the fourth application of this invention, a recombinant form of the Abelson murine leukaemia virus protein tyrosine kinase v-Abl was used in an in vitro phosphorylation reaction to phosphorylate the library sub-set 4 which scans the sequence −1 to +3 around a zero position tyrosine residue, as previously performed for the ZAP-70 library. Detection of phosphoryrosine was achieved using anti-phosphotyrosine antibody detection in an ELISA assay using tetramethylbenzidine substrate and recording absorbance at 450 nm. Background absorbance readings of 0.11 were recorded while the highest substrate peptide value was 0.32. Deconvolution of the hit peptides was performed as described in WO 97/42216. Clear defined substrates were deconvoluted in the library sub-set.

In the fifth application of this invention, the invention was used to map the substrate specificity of a protein serine or serine/threonine kinase (which include I-kappa B kinase beta and cAMP-dependent protein kinase [cAPK]). A protein serine or serine/threonine kinase enzyme was used in an in vitro phosphorylation reaction to phosphorylate the five substrate sub-libraries which scan the sequence −4 to −4 around a central serine residue. The library was synthesised as the protein tyrosine kinase ZAP-70 library save that the tyrosine faced residues were replaced with a serine which was then scanned through the five sub-libraries. Detection of phosphoserine was achieved using anti-phosphoserine antibody detection in an ELISA assay using tetramethylbenzidine substrate and recording absorbance at 450 nm. Deconvolution of the hit peptides was performed as described in WO 97/42216.

General formula: (Xaa)ₓSer (Xaa)_{y}[SEQ ID No. 4].

Library Sub-Set 1 Xaa-Xaa-Xaa-Xaa-Ser

Library Sub-Set 2 Xaa-Xaa-Xaa-Ser-Xaa

Library Sub-Set 3 Xaa-Xaa-Ser-Xaa-Xaa

Library Sub-Set 4 Xaa-Ser-Xaa-Xaa-Xaa

Library Sub-Set 5 Ser-Xaa-Xaa-Xaa-Xaa

Likewise the Library Sub-Sets can be synthesised for the sapping of threonine kinases by the synthesis of a library containing the threonine residue to allow phosphorylation by enzymes recognising this residue.

It will be realised by those skilled in the art that the replacement of the recognition residue such as the tyrosine, or serine, with a residue that is covalently modified by the enzyme to be mapped allows the active site of any such enzyme to be determined according to the invention.

The invention will now be described by reference to the following examples.

### Example 1

In this example the invention was used to map the active catalytic site of ZAP-70, a protein kinase enzyme that catalyses the phosphorylation of a tyrosine residue. The example illustrates the synthesis of a number of compounds, and their use as a sub-set library for the mapping of the enzyme so as to allow the subsequent identification and synthesis of single specific substrates for the enzyme.

Synthesis of Peptide Compounds.

Preparation of Crown Assembly

The peptide compounds were synthesised in parallel fashion using Fmoc-Rink-DA/MDA derivatised gears (ex Chiron Mimotopes, Australia) loaded at approximately 1.6 μM per crown. Prior to synthesis each crown was connected to its respective stem and slotted into the 8×12 stem holder. Coupling of the amino acids employed standard Fmoc amino acid chemistry as described in ‘Solid Phase Peptide Synthesis’, E. Atherton and R. C. Sheppard, IRL Press Ltd, Oxford, UK, 1989.

Removal of N-Fmoc Protection

A 250 ml solvent resistant bath is charged with 200 ml of a 20% piperidine/DMF solution. The multipin assembly is added and deprotection allowed to proceed for 30 minutes. The assembly was removed and excess solvent removed by brief shaking. The assembly is then washed consecutively with (200 ml each), DMF (5 minutes) and MeOH (5 minutes, 5 minutes, 5 minutes) and left to air dry for 15 minutes.

Quantitative UV Measurement of Fmoc Chromophore Release

A 1 cm path length UV cell is charged with 1.2 ml of a 20% piperidine/DMF solution and used to zero the absorbance of the UV spectrometer at a wavelength of 290 nm. A UV standard is then prepared consisting of 5.0 mg Fmoc-Asp(OBut)-Pepsyn KA (0.08 mmol/g) in 3.2 ml of a 20% piperidine/DMF solution. This standard gives Abs₂₉₀=0.55-0.65 (at room temperature). An aliquot of the multipin deprotection solution is then diluted as appropriate to give a theoretical Abs₂₉₀=0.6, and this value compared with the actual experimentally measured absorbance showing the efficiency of previous coupling reaction.

Coupling of Standard Amino Acid Residues

Coupling reactions were performed by charging the appropriate wells of a polypropylene 96 well plate with the pattern of activated solutions required during a particular round of coupling. Gear (approx 1.6 μmole) standard couplings were performed in DMF (300 μl).

Coupling of an Amino-acid Residue to Appropriate Well

Whilst the multipin assembly is drying, the appropriate N-Fmoc amino acid pfp esters (10 equivalents calculated from the loading of each crown) and HOBt (10 equivalents) required for the particular round of coupling are accurately weighed into suitable containers. Alternatively, the appropriate N-Fmoc amino acids (10 equivalents calculated from the loading of each crown), desired coupling agent e.g. HBTU (9.9 equivalents calculated from the loading of each crown) and activation e.g. HOBt (9.9 equivalents calculated from the loading of each crown), NMM (19.9 equivalents calculated from the loading of each crown) were accurately weighed unto suitable containers.

The protected and activated Fmoc amino acid derivatives were then dissolved in DMF (300 l for each gear e.g. for 20 gears, 20×10 eq.×1.6 μmoles of derivative would be dissolved in 10 ml DMF). The appropriate derivatives were then dispensed to the appropriate wells ready for commencement of the ‘coupling cycle’. As a standard, coupling reactions were allowed to proceed for 6 hours. The coupled assembly was then washed as detailed below.

Coupling of d-Biotin Acid to Pins

d-Biotin (10 eq), 1-hydroxybenzotriazole.H₂O (10 eq), BOP (9.95 eq) and NMM (19.9 eq) were dissolved in DMF (0.3mL per well) and agitated for 2 minutes. 300 μL of solution was dispensed to each well of a 96-well polypropylene plate. The gears were then added to the solution and left for 24 hours. Fresh solution was made up, the gears washed in DMF for 5 minutes and then added to the fresh coupling mixture and left a further 24 hours.

The pin assembly was removed from the plate, shaken free of excess liquid then immersed in DMF (200 mL) for 5 mins. The assembly was again shaken then immersed in MeOH (200 mL, 3×5 mins) and allowed to air dry.

Washing Following Coupling

If a 20% piperidine/DMF deprotection is to immediately follow the couplincg cycle, then the multipin assembly is briefly shaken to remove excess solvent washed consecutively with (200 ml each), MeOH (5 minutes) and DMF (5 minutes) and de-protected (see 6.2). If the multipin assembly is to be stored or reacted further, then a full washing cycle consisting brief shaking then consecutive washes with (200 ml each), DMF (5 minutes) and MeOH (5 minutes, 5 minutes, 5 minutes) is performed.

Following these general methods, the peptide libraries shown in FIG. 1 were sequentially assembled by applying the appropriate coupling procedure at the correct cycle during synthesis.

Acidolytic Mediated Cleavage of Peptide-Pin Assembly

Acid mediated cleavage protocols were strictly performed in a fume hood. A polystyrene 96 well plate (1 ml/well) was labelled, then the tare weight measured to the nearest mg. Appropriate wells were charged with a trifluoroacetic acid/triisopropylsilane (95:5, v/v, 600 μl) cleavage solution, in a pattern corresponding to that of the multipin assembly to be cleaved.

The multipin assembly is added, the entire construct covered in tin foil and left for 2 hours. The multipin assembly in then added to another polystyrene 96 well plate (1 ml/well) containing trifluoroacetic acid/triethylsilane (95:5, v/v, 600 μl) (as above) for 5 minutes.

Work Up of Cleaved Peptides

The primary polystyrene cleavage plate (2 hour cleavage) and the secondary polystyrene plate (5 minute wash) were then placed in the SpeedVac and the solvents removed (minimum drying rate) for 90 minutes.

The contents of the secondary polystryene plate were transferred to their corresponding wells on the primary plate using an acetonitrile/water/acetic acid (50:45:5, v/v/v) solution (3×150 μl) and the spent secondary plate discarded.

Analysis of Products

A 5 μL aliquot from each well is diluted to 100 μl with 0.1% aq. TFA, then a 10 μL aliquot from this plate diluted with a further 100 μl 0.1% aq. TFA. The double diluted plate was analysed by HPLC-MS.

Final Lyophilisation of Peptides

The plate was covered with tin foil, held to the plate with an elastic band. A pin prick was placed in the foil directly above each well and the plate placed at −80° C. for 30 minutes. The plate was then lyophilised on the ‘Heto freeze drier’ overnight. Finally, the dried plate was weighed. The total cleaved peptide was quantified (by weight) and the average content of each peptide calculated. Since all the peptides present have originated from the same peptide-pin assembly, cleaved under identical conditions, it is reasonable to assume that the contents of each well are roughly equimolar.

Protein Kinase Cloning, Expression and Purification

Polymerase Chain Reaction (PCR) and Downstream Cloning

The coding sequence for human ZAP-70 amino acid 306-615 was amplified from Jurkat T cell cDNA by PCR (2 minutes at 94° C., followed by 35 cycles of 15 seconds 94° C., 30 seconds 65° C., 2 minutes 72° C. and a final single 5 minute 72° C. incubation) using the primers:

| | |
|---|---|
| 5′ CCGGGATCCGCCATGCCCATGGACACGAGCGTGTAT 3′ | [SEQ ID No. 5] |
| 5′ GGGGGATCCTCAGTGGTGGTGGTGGTGGTGGGCACAGGCAGCCTCAGC | [SEQ ID No. 6] |
| CTTCTGTGT 3′ | |

The PCR amplicon was cloned into the Bam HI site of pUC19 and sequence confirmed using M13-20 and reverse primers on an Applied Biosystems Prism 310 sequencer as described by manufacturer. The Bam HI ZAP-70 insert was excised from the sequencing vector and ligated into the Baculoviral transfer vector pAcUW51 (Pharmingen).

Generation of ZAP-70 Enzyme Using Baculovirus

Homologous recombination with wild type baculoviral DNA was then performed in Sf9 insect cells and viral supernatant harvested. Plaque purified virus was exposed to several viral amplification steps then used at a titre of 3×10⁹ PFU/ml to infect 3 l 1×10⁶ cells/ml Sf9 cells at an MOI of 10 in an Applecon bioreactor using 60% dissolved oxygen. Cells were harvested 3 days post infection.

Protein Purification

The infected cell pellet was lysed in 50 mM Tris-HCl, pH 7.5, 0.15 M NaCl, 25% sucrose, 1 mM 4-nitrophenol phosphate, 1 mM sodium orthovanadate and protease inhibitors.

Following homogenisation using a dounce pestle B, the cleared lysate was loaded onto a cobalt-sepharose column. After column washing with lysis buffer, elution was performed with an imidazole gradient and ZAP-70 fractions identified by protein kinase activity against the peptide substrate Lys-Lys-Lys-Lys-Ala-Asp-Glu-Glu-Asp-Tyr-Phe-Ile-Pro-Pro-Ala as described in Casnellie et al, 1991.

Library Screening

Library peptides were phosphorylated in pools of 20 peptides at a final concentration of 1 μM total peptide in 50 mM HEPES, pH 7.5, 0.1% Triton X-100, 100 μM ATP, 10 mM MnCl₂, 1 mM DTT and 0.2 mM sodium orthovanadate for 30 minutes at 30° C. These reaction mixtures were then stopped using 100 mM EDTA, 6 mM adenosine, transferred to strepavidin-coated microtiter plates and allowed to bind for 30 minutes at 20° C. Unincorporated reaction products were washed from the plate using PBS/0.1% Tween 20 then plate incubation performed with anti-phosphotyrosine antibody (Sigma mouse monoclonal clone PT66) in 2% BSA/PBS/Tween for 1 hour at 20° C. Unbound antibody was removed by plate washing using PBS/Tween then incubation performed with rabbit anti mouse-HRP (Amersham) for a further 1 hour. HRP detection was then performed with tetramethylbenzidine (TMB) substrate and measuring absorbance at 450 nM using a spectrophotometer (Dynex MRX).

The best substrates were identified as those which gave the highest amount of phosphate incorporation. The library subsets were deconvoluted according to the teaching of WO097/42216: this gives an immediate determination of the unique sequence of any phosphorylated motif without the need for further synthesis or sequencing. (FIG. 2 [SEQ ID Nos. 7, 8, 9, 10]).

Peptide Kₘ Determination

Biotin-tagged peptides were phosphorylated at varying concentrations in 50 mM HEPES, pH 7.5, 0.1% Triton X-100, 200 μM ATP, 10 mM MnCl₂, 1 mM DTT and 0.2 mM sodium orthovanadate using 0.5 μCi ³³P-γ-ATP for 10 minutes at 30° C. The reactions were stopped using 2 M guanidine hydrochloride, diluted 1 in 10 in water then 5 μl reaction mix spotted onto SAM™ titre plates (Promega). Unincorporated reaction products were washed as described by manufacturer then 20 μl scintillation liquid added and plate counted on a Packard TopCount beta-counter. Kₘ was calculated using a non-linear one site hyperbola model (ATP was added in excess to negate influence of the ATP binding site on the substrate site kinetics). (FIG. 3).

### EXAMPLE 2

In this example the invention was used to map the active catalytic site of Syk, a protein kinase enzyme that catalyses the phosphorylation of a tyrosine residue. The example illustrates the positional scanning of the sub-set libraries for the mapping of the enzyme, and their use so as to allow the subsequent identification of the preferred substrates of the enzyme catalytic site.

The mapping and assessment of the catalytic site was performed as detailed in Example 1. The substrate preferences were deconvoluted as detailed in WO 97/42216 and are detailed below.

Library Sub-Set 1 Asp-Glu-Glu-Asp-Tyr [SEQ ID No. 11]

Library Sub-Set 2 Asp-Glu-Glu-Tyr-Asp [SEQ ID No. 12]

Library Sub-Set 3Asp-Glu-Tyr-Glu-Asp [SEQ ID No. 13]

Library Sub-Set 4 Asp-Tyr-Glu-Glu-Val [SEQ ID No. 14]

Library Sub-Set 5 Tyr-Ser-Ile-Ile-Nle [SEQ ID No. 15]

### EXAMPLE 3

In this example the invention was used to map the active catalytic site of CSK. The subset library was used to scan the enzyme active site so as to allow the subsequent identification and synthesis of the preferred specific substrates for the enzyme as listed below.

Library Sub-Set 1 Asp-Glu-Glu-Glu-Tyr [SEQ ID No. 16]

Library Sub-Set 2 Asp-Glu-Glu-Tyr-Phe [SEQ ID No. 17]

Library Sub-Set 3 Asp-Glu-Tyr-His-Asn [SEQ ID No. 18]

Library Sub-Set 4 Asp-Tyr-His-Leu-Phe [SEQ ID No. 19]

Library Sub-Set 5 Tyr-Pro-Ile-Glu-Val [SEQ ID No. 20]

### EXAMPLE 4

In this example the invention was used to map the active catalytic site of v-Abl, a protein kinase enzyme that catalyses the phosphorylation of a tyrosine residue. For this enzyme only the Library Sub-Set 4 (i.e. X-Tyr-X-X-X according to SEQ ID No. 2) was scanned with the enzyme. The active site substrate recognition substrate for this enzyme for this Sub-Set was Serine-tyrosine-phenylalanine-histamine-glutamine [SEQ ID No. 21].

### REFERENCES

Arpaia E, Shahar M, Dadi H, Cohen A and Roifman C M. (1994). Defective T cell receator signaling and CD8+ thymic selection in humans lacking zap-70 kinase. Cell 76(5):947-958

Barker K T, Jackson L E, and Crompton M R. (1997). BRK tyrosine kinase expression in a high proportion of human breast carcinomas. Oncogene 14; 15(7):799-805

Casnellie, J E. (1991). Assay of protein kinases using peptides with basic residues for phosphocellulose binding. Methods Enz. 200, 115-120.

Denti, L., Vetriani, C., Zucconi, A., Pelicci. G., Lanfrancone, L., Pelicci, P. G. and Cesareni, G. (1997). Modified phage peptide libraries as a tool to study specificity of phoschorylation and recognition of tyrosine containing peptides. J. Mol. Biol., 269, 694-703.

Elder M E, Lin D, Clever J, Chan A C, Hope T J, Weiss A, Parslow T G. (1994). Human severe combined immunodeficiency due to a defect in ZAP-70, a T cell tyrosine kinase. Science 264(5165):1596-1599

Hanks, S K and Quinn, M. (1991). Protein kinase catalytic domain sequence database: identification of conserved features of primary structure and classification of family members. Methods Enzymol 200, 38-62

Kemp, B E, Pearson, R B, and House, C M, (1991). Pseudosubstrate-based peptide inhibitors. Methods Enzymol 201, 287-304.

Lee J C, Lavdon J T, McDonnell P C, Gallazker T F, Kumar S, Green D, McNulty D, Blumenthal M J, Heys J R, Landvatter S W, et al. (1994). A protein kinase involved in the regulation of inflammatory cytokine biosynthesis. Nature 372(6508):739-746

Lehtola L. Partanen J, Sistone L, Korhonen J, Warri A., Harkonen P, Clarke R and Alitalo K. (1992). Analysis of tyrosine kinase mRNAs including four FGF receptor mRNAs expressed in MCF-7 breast-cancer cells. Int J Cancer 50(4):598-603

Roifman C M. (1995). A mutation in zap-70 protein tyrosine kinase results in a selective immunodeficiency. J Clin Immunol 15(6 Suppl):52S-62S

Schmitz, R., Baumann, G. and Gram, H. (1996). Catalytic specificity of phosphotyrosine kinases Blk. Lyn, c-Src and Syk as assesses by phae display. J. Mol. Biol., 60, 664-677. Songyang Z, Blechner S, Hoagland N, Hoekstra M F, Piwnica-Worms H and Cantley L C. (1994). Use of an oriented peptide library to determine the optimal substrates of protein kinases. Curr Biol. 4(11):973-982

Wardenburg, J. B., Fu., C., Jackman, J. K, Flotow, H., Wilknson, S. E., Williams, D. H., Johnson, R., Kong, G., Chan, A. C. and Findell, P. R. (1996). Phosphorylation of SLP-76 by the ZAP-70 protein-tyrosine kinase is required for T-cell receptor function. J. Biol. Chem. 271(33), 19641-19644.

Wilson K P, Fitzgibbon M J, Caron P R, Griffith J P, Chen W, McCaffry P G, Chambers S P and Su M S. (1996). Crystal structure of p38 mitogen-activated protein kinase. J. Biol Chem. 271(44):27696-27700

Xu W, Harrison S C and Eck M J. (1997). Three-dimensional structure of the tyrosine kinase c-Src. Nature 385(6617):595-602

Zhang F, Strand A, Robbins D, Cobb M H and Goldsmith E J. (1994) Atomic structure of the MAP kinase ERK2 at 2.3 A resolution. Nature 367(6465):704-711

1. A method for determining an amino acid sequence motif or a peptidomimmetic sequence motif containing an active site capable of being bound by an enyyme which catalyses covalent modification of a substrate molecule, comrprising;
a) contagting the enzyme with a library consisting of a number of oriented degenerate library subsets of molecules, each subset comprising unmodified degenerate motif sequences each having n residues and each having a modifiable residue at a different fixed non-degenerate position, under conditions which allow for modification of molecules which are a substrate for the enzyme;
b) allowing the enzyme to modify modifiable residues in library subsets containing molecules having an active substrate site for the enzyme;
c) deconvoluting the oriented degenerate library subsets of the library, in situ without separating modified from unmodified molecules, so as to reveal the sequence of any motif which has been modified by covalent binding of the enzyme;
wherein each library subset is of formula (I)
(Xaa)ₓ Zaa (Xaa)_{y} (I) [SEQ ID No. 1]
wherein
Zaa is a non-degenerate modifiable natural or unnatural amino acid residue or peptidomimetic;
Xaa is any natural or unnatural ammo acid residue or peptidomiinetic;
x and y are each independently 0 or an integer;
(x+y)=(n−1); and
n=an integer from 3 to 8, preferably 5.

2. A method according to claim 1 which includes the further step of synthesising a substrate molecule containing a motif sequence revealed in step (c) or an analogue of said motif sequence.

3. A method according to claim 1 in which said revealed substrate molecule motif sequence, or an analogue thereof, is used to develop a selective inhibitor of said enzyme, which method includes the step of changing the modifiable residue to a derivative form of the residue which is not modifiable by the enzyme.

4. An enzyme substrate molecule produced according to the method of claim 2.

5. An enzyme inhibitor molecule produced according to the method of claim 3.

6. A pharmaceutical composition comprising as an active ingredient a substrate molecule according to claim 2.

7. A pharmaceutical composition comprising as an active ingredient an inhibitor molecule according to claim 3.

8. A method of treatment which includes administering to a patient an effective amount of a substrate molecule according to claim 2 or a composition according to claim 6.

9. A method of treatment which includes administering to a patient an effective amount of an inhibitor molecule according to claim 3 or a composition according to claim 7.

10. A method according to claim 1 wherein x+y=(n−1)=4.

11. A method according to claim 1 or 10 wherein the step (c) of deconvolution is carried out according to the procedure for auto-deconvolution of combinatorial libraries described in WO 97/42216.

12. A method according to claim 1 wherein Formula 1 may optionally include at any place in the formula one or more invariant residue(s), said residue(s) being in the same relative position(s) in each subset of the library.

13. A method according to any of claims 1 to 3 and 8 to 12 wherein said enzyme catalyses covalent modification selected from the group consisting of phosphorylation, acylation; and dephosphorylation.

14. A method according to any of claims 1 to 3 and 8 to 13 wherein said enzyme is a protein kinase enyyme.

15. A method according to any of claims 1 to 3 and 8 to 14 wherein said modifiable residue Z is selected from the group consisting of tyrosine; serine; threonine; histidine; and aspartic acid.

16. A protein kinase inhibitor capable of inhibiting the catalytic transfer of the γ-phosphate from ATP to an amino acid residue on a substrate molecule, said inhibitor having been produced by the method of any of claims 1 to 3 and 8 to 15.
